(19) United States
(12) Patent Application Publication
(10) Pub. No.: US 2003/0060839 A1
(43) Pub. Date: Mar. 27, 2003

(54) Corneal surgical apparatus

(75) Inventor: Masahiro Sugimura, Aichi (JP)

Correspondence Address: Finnegan, Henderson, Farabow,, Garrett & Dunner, L.L.P., 1300 I Street, N.W., Washington, DC, 20005-3315, US

(73) Assignee: Nidek Co., Ltd.

(21) Appl. No.: 10/226,124

(22) Filed: Aug. 23, 2002

(30) Foreign Application Priority Data
Aug. 24, 2001 (JP) P2001-255291

(51) Int. Cl. A61F 9/00

(52) U.S. Cl. 606/166

(57) ABSTRACT

A corneal surgical apparatus for incising a cornea of a patient's eye in a layered form, includes; a rotating shaft; and a bearing rotatably supporting the rotating shaft. At least one of a surface of the rotating shaft and an inner wall surface of the bearing has been subjected to a coating treatment or a surface modification treatment.

### BACKGROUND OF THE INVENTION

1. Field of the Invention

The present invention relates to a corneal surgical apparatus for incising the cornea of an eye of a patient in a layered form at the time of a keratorefrative surgery or the like.

2. Description of the Related Art

In recent years, attention has been focused on a LASIK (laser in situ keratomileusis) surgery for the keratorefractive surgery wherein a flap is formed by incising a corneal portion with a thickness of about 150 μm (0.15 mm) ranging from the corneal *epithelium to the corneal stroma with a part of the cornea remaining connected like a hinge, ablating the corneal stroma in a refractive correction amount by an excimer laser light, and returning the flap to its original position. In the LASIK surgery, a corneal is surgical apparatus called a microkeratome is used for incising the cornea in a layered form.

As a corneal surgical apparatus, one comprising a suction ring to be vacuum-fixed to a part of the cornea from a corneal ring portion to the surface of the conjunctiva, a cornea applanating member for applanating the cornea flatly, and a blade movable toward the hinge while being oscillated laterally so as to incise the flattened cornea into a layer form with a substantially uniform thickness, is known.

As a mechanism for the blade lateral oscillation, one comprising a motor, a rotating shaft to be rotated by the motor, a bearing rotatably supporting the rotating shaft, and a transmitting member, such as an eccentric pin, for converting the rotation of the motor transmitted through the rotating shaft into the lateral oscillation to be transmitted to the blade, is proposed. To incise the cornea uniformly and easily, the blade is required to be oscillated laterally at a high speed, and thus the motor rotates the rotating shaft at a high speed.

However, moisture, such as eye drops (physiological salt solution) to be dropped into the patient's eyes or tears, sometimes intrudes into a nominal clearance between the rotating shaft and the bearing during surgery, thereby rendering the rotating shaft and the-bearing susceptible to rust. When the rotating shaft is rotated, metal contact arises between the rotating shaft and the bearing, whereby adhesion becomes likely to arise. Occurrence of rust or adhesion of metal becomes an influential factor for impeding rotation of the shaft.

In light of the drawbacks of the related art, the invention is aimed at providing a corneal surgical apparatus which inhibits occurrence of rust or adhesion of metal by virtue of a simple construction and which is capable of stably rotating a rotating shaft.

### SUMMARY OF THE INVENTION

The present invention provides the following arrangements:

(1) A corneal surgical apparatus for incising a cornea of a patient's eye in a layered form, comprising:

a rotating shaft; and

a bearing rotatably supporting the rotating shaft,

wherein at least one of a surface of the rotating shaft and an inner wall surface of the bearing has been subjected to a coating treatment or a surface modification treatment.

(2) The apparatus of (1), wherein the coating treatment includes a DLC coating treatment.

(3) The apparatus of (2), wherein the coating treatment applies a film having a thickness of 0.5 to 1.5 μm onto the at least one of the rotating shaft surface and the bearing inner wall surface.

(4) The apparatus of (1), wherein the coating treatment applies a film having a friction coefficient equal to or less than 0.7 onto the at least one of the rotating shaft surface and the bearing inner wall surface.

(5) The apparatus of (1), wherein the surface modification treatment includes a carbon solution diffusion processing,

(6) The apparatus of (1), wherein at least one of the rotating shaft and the bearing is made of stainless steel.

(7) A corneal surgical apparatus for incising a cornea of a patient's eye in a layered form, comprising:

a rotating shaft: having a surface that has been subjected to a DLC coating treatment; and

a bearing rotatably supporting the rotating shaft, the bearing having an inner wall surface that has been subjected to a surface modification treatment.

The present disclosure relates to the subject matter contained in Japanese patent application No. 2001-255291 (filed on Aug. 24, 2001), which is expressly incorporated herein by reference in its entirety.

### BRIEF DESCRIPTION OF THE DRAWINGS

In the accompanying drawings:

FIGS. 1(a) and 1(b) show a top view and a cross-sectional view of a corneal surgical apparatus of an embodiment; and

FIG. 2 is a schematic view of a rotating shaft, an eccentric pin, and a connecting member.

### DESCRIPTION OF THE PREFERRED EMBODIMENT

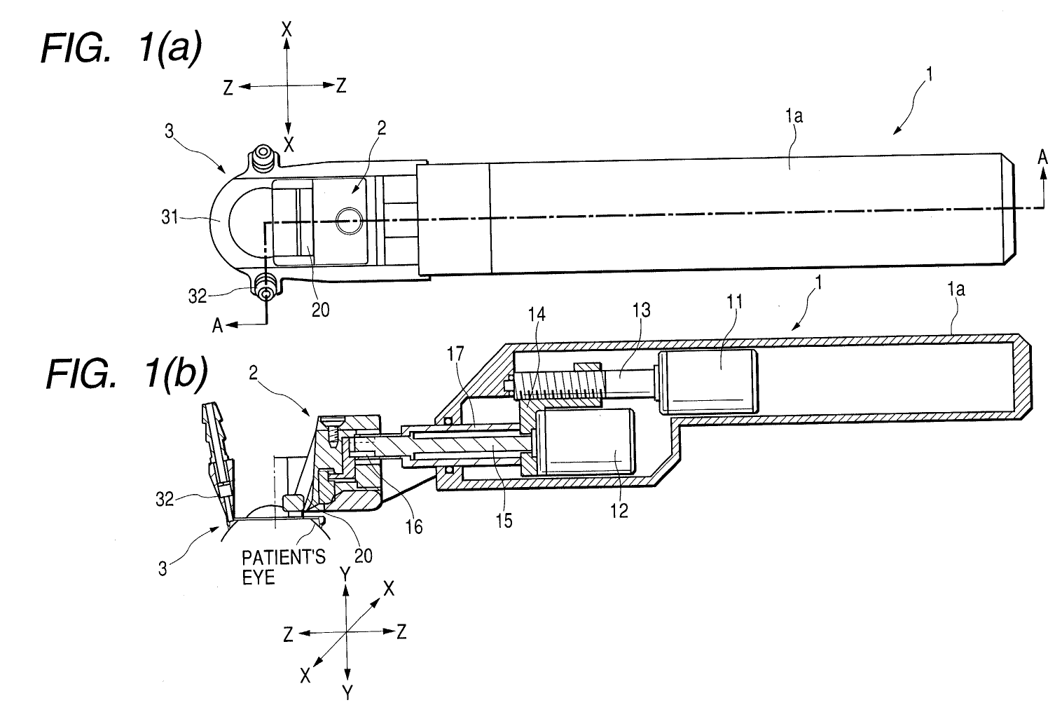

Referring to the accompanying drawings, a description will be given of an embodiment of the present invention. FIG. 1(a) is a top view of a corneal surgical apparatus according to the embodiment of the present invention. FIG. 1(b) is a cross-sectional view taken along a line A-A of FIG. 1(a).

Reference numeral 1 denotes a main body of the corneal surgery apparatus (microkeratome), and reference numeral 1a denotes a grip to be held by an operator during the surgery. A suction unit 3 for fixing the main body 1 to the patient's eye and a cutting unit 2, which has a blade 20 for incising the cornea and is adapted to move rectilinearly (translate) on the suction unit 3, are provided on the front side (left-hand side in the drawing) of the main body 1.

A translation motor 11 for rectilinearly moving (translating) the cutting unit 2 (the blade 20) in the incising direction (in the Z direction) and an oscillation motor 12 for imparting oscillations in the lateral direction (in the X direction) to the blade 20 are installed in the main body 1. A translation screw 13 is coupled to a rotating shaft of the motor 11, which has a threaded portion corresponding in length to the rectilinear movement (translation) or travel of the cutting unit 2. An attaching member 14 is threadedly engaged with the screw 13. The motor 12 as well as a connecting member 17 to which the cutting unit 2 is connected are fixed to the attaching member 14. As the motor 11 is rotated forwardly or reversely, the motor 12 and the connecting member 117 move (translate) forwardly or backwardly (in the Z direction) through the screw 13 and the attaching member 14, thereby causing the cutting unit 2 to move (translate) forwardly or backwardly. The motors 11 and 12 are driven under a control by a control unit (not-shown).

Further, the connecting member 17 serves also as a bearing or rotational support for a rotating shaft 15 so that the rotating shaft 15 is rotatably held by the connecting member 17. An eccentric pin 16 is embedded or protruded on a distal end of the rotating shaft 15 at a position offset from the center of rotation. The shaft 15 and the pin 16 impart lateral oscillations to the blade 20 (see EP0956840A2 for details). A metal blade having a blade edge of stainless steel, steel, or the like or a mineral blade having a blade edge of diamond, sapphire or the like is used as the blade 20.

Reference numeral 31 denotes a suction ring for stably holding the main body 1 onto patient's eye, and reference numeral 32 denotes a suction pipe. The suction pipe 32 is connected to a pump (not shown) so that the suction ring 31 is vacuum-fixed to the patient's eye by vacuum action of the pump.

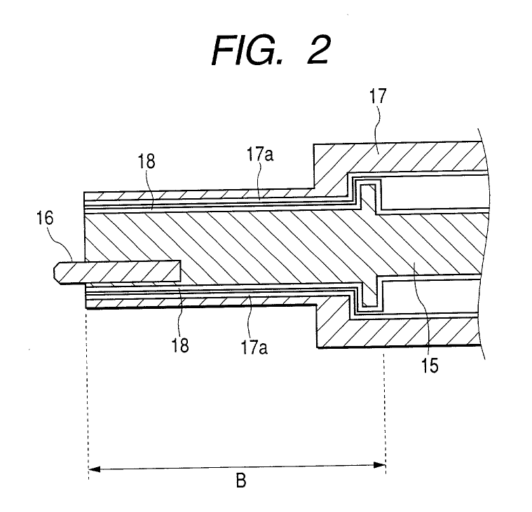

FIG. 2 is a view showing schematic construction of the rotating shaft 15, the eccentric pin 16, and the connecting member 17.

In the drawing, reference symbol B designates an area in which the connecting member 17 retains (holds) the shaft 15. In the area B, the shaft 15 and the connecting member 17 are preferably in intimate contact with each other to the extent possible, in order to prevent intrusion of moisture into the inside of the connecting member 17 while the shaft 15 is being rotatably supported. In the embodiment, stainless steel is used for the shaft 15 and the connecting member 17, in view of resistance to rusting and ease of processing. In addition to the stainless steel, a metal material having a superior anti-corrosion characteristic such as an aluminum alloy processed with alumite or a titanium alloy can also be used.

An interior wall surface 17a of the connecting member 17 is subjected to carbon solution diffusion processing (NV pionite treatment, AIR WATER INC.) as surface modification treatment. The thus-treated interior wall surface 17a has a surface roughness of Hv 800 or thereabouts and a superior anti-corrosion characteristic. Such treatment forms an interior wall surface which maintains the property of stainless steel and is resistant to abrasion and rust.

In the embodiment, the interior wall surface 17a of the connecting member 17 is subjected to carbon solution diffusion treatment. However, the invention is not limited to this treatment; any surface modification treatment, such as nitriding treatment, may be performed, so long as the treatment is a surface modification treatment which increases a surface hardness of the interior surface and imparts a superior anti-corrosion characteristic. Moreover, there may be employed coating treatment which increases surface hardness and imparts a superior anti-corrosion characteristic and impact resistance.

The surface of the shaft 15 is subjected to DLC (diamond-like carbon) coating treatment, and a DLC layer 18 is formed on the shaft 15. The DLC coating treatment is performed by means of an ion plating method, which is a process involving plasma in a high vacuum. The DLC layer 18 has a thickness of 0.5 to 1.5 μm. The DLC layer 18 has a surface roughness of Hv 1000 to 5000 or thereabouts, thereby improving the anti-abrasion characteristic, anti-corrosion characteristic, and non-adhesion characteristic of the shaft 15. Moreover, the friction coefficient of the DLC layer 18 is reduced to a value of 0.1 or less by means of DLC coating treatment. Hence, friction developing between the shaft 15 and the connecting member 17 is diminished. By means of such a configuration, occurrence of rust or adhesion of metal can be inhibited.

The embodiment employs the DLC coating. However, the invention is not limited to the DLC coating; any coating method may be employed, so long as a friction coefficient of a film stemming from coating assumes a value of about 0.7 or less. Kinds of films to be produced include, among others, a CrN film a TiN film, a TiCN film, a TiC film, and a TiAlN film.

In the embodiment, the shaft 15 is subjected to DLC coating treatment, and the connecting member 17 is subjected to NV pionite treatment. However, the invention is not limited to these treatment methods. If the shaft 15 is subjected to DLC coating treatment, the connecting member 17 does not need to be subjected to NV pionite treatment. In contrast, if the connecting member 17 is subjected to NV pionite treatment, the shaft 15 does not need to be subjected to DLC coating treatment.

Further, the shaft 15 may be subjected to NV pionite treatment. In this case, the connecting member 17 does not need to be subjected to the same type of treatment (i.e., NV pionite treatment).

### EXAMPLE 1

In Example 1, SUS 316 (conforming with JIS), which is a kind of stainless steel and has been subjected to DLC coating treatment, was used for the shaft 15. Further, SUS 316 having been subjected to NV pionite treatment was used for the connecting member 17. A drive test and a salt water test were carried out as evaluation tests.

The drive test was performed in the following manner.

In order to realize a situation encountered in actual usage, a blade 20 of a corneal surgical apparatus was immersed in physiological salt solution, and the shaft 15 was rotated at a speed of 9,000 rpm or thereabouts. A cutting unit 2 (blade 20) was moved (translated) back and forth one time over a suction ring 31 for 10 seconds, and then halted for 20 seconds. One cycle is constituted by combination of oscillation and translation of the blade 20 for 10 seconds and halting of the blade 20 for 20 seconds. The test was performed 10,000 times (10,000 cycles) and 20,000 times (20,000 cycles). Then, drive states and appearance of contact surfaces of the shaft 15 and the connecting member 17 were evaluated. A lubricant was applied between the shaft 15 and the connecting member 17.

As the salt water test, the shaft 15 was immersed in a physiological salt solution, and changes having arisen in the surface state of the shaft 15 after lapse of time (i.e., after lapse of five hours, lapse of one day, lapse of one week, and lapse of two weeks) were visually evaluated.

### EXAMPLE 2

In Example 2, SUS 316 having been subjected to DLC coating treatment was used for the shaft 15. SUS 316 having not been subjected to any treatment was used for the connecting member 17. As in the case of Example 1, a lubricant was applied between the shaft 15 and the connecting member 17, and the drive test was performed. Further, a salt water test analogous to that performed in connection with Example 1 was also performed.

### EXAMPLE 3

In Example 3, SUS 316 that has been subjected to NV pionite treatment was used for the shaft 15. SUS 316 having not been subjected to any treatment was used for the connecting member 17. As in the case of Example 1, a lubricant was applied between the shaft 15 and the connecting member 17, and the drive test was performed. Further, a salt water test analogous to that performed in connection with Example 1 was also performed.

### COMPARATIVE EXAMPLE 1

In Comparative Example 1, SUS 420 having been subjected to hardening treatment was used for the shaft 15. SUS 316 having not been subjected to any treatment was used for the connecting member 17. As in the case of Example 1, a lubricant was applied between the shaft 15 and the connecting member 17, and the drive test was performed. Further, a salt water test analogous to that performed in connection with Example 1 was also performed.

### COMPARATIVE EXAMPLE 2

In Comparative Example 2, SUS 316 having not been subjected to any treatment was used for the shaft 15 and the connecting member 17. As in the case of Example 1, a lubricant was applied between the shaft 15 and the connecting member 17, and the drive test was performed. Further, a salt water test analogous to that performed in connection with Example 1 was also performed.

Table 1 shows results of Examples 1 through 3 and those of Comparative Examples 1 and 2.

TABLE 1

| | | | | | COMPARATIVE | COMPARATIVE |
|---|---|---|---|---|---|---|
| | | EXAMPLE 1 | EXAMPLE 2 | EXAMPLE 3 | EXAMPLE 1 | EXAMPLE 2 |
| MATERIAL | SUS 316 (HAVING BEEN | ◯ | ◯ | | | |
| (OF SHAFT) | SUBJECTED | | | | | |
| | TO DLC TREATMENT) | | | | | |
| | SUS 316 (HAVING BEEN | | | ◯ | | |
| | SUBJECTED TO NV | | | | | |
| | PIONITE TREATMENT) | | | | | |
| | SUS420 | | | | ◯ | |
| | SUS316 | | | | | ◯ |
| MATERIAL | SUS 316 (HAVING | ◯ | | | | |
| (OF BEARING) | BEEN SUBJECTED | | | | | |
| | TO NV | | | | | |
| | PIONITE TREATMENT) | | | | | |
| | SUS316 | | ◯ | ◯ | ◯ | ◯ |
| DRIVE TEST | 10,000 TIMES | ⊚ | ⊚ | ⊚ | ◯ | X |
| | 20,000 TIMES | ⊚ | ⊚ | ⊚ | Δ | |
| SALT WATER | AFTER LAPSE OF | ⊚ | ⊚ | ⊚ | X | ⊚ |
| TEST | FIVE HOURS | | | | | |
| | AFTER LAPSE OF | ⊚ | ⊚ | ⊚ | X | ⊚ |
| | ONE DAY | | | | | |
| | AFTER LAPSE OF | ⊚ | ⊚ | ⊚ | X | ⊚ |
| | ONE WEEK | | | | | |
| | AFTER LAPSE OF | ⊚ | ⊚ | ⊚ | X | ⊚ |
| | TWO WEEKS | | | | | |
| OVERALL | | ⊚ | ⊚ | ⊚ | X | X |
| EVALUATION | | | | | | |

As shown in Table 1, in Examples 1 and 2, a decrease in the number of rotations or a halt of rotation stemming from adhesion of metal never arose even when the shaft 15 and the connecting member 17 were subjected to a drive test 20,000 times. Moreover, no changes in the appearance of contact surfaces of the shaft 15 and the connecting member 17 were observed. In relation to the salt water test, no rust was observed to have arisen in the surface of the shaft 16 even after lapse of two weeks (2W).

In Example 3, a superior result was obtained as a result of the shaft 15 and the connecting member 17 having been subjected to the drive test 10,000 times. However, a slight change arose in the appearance of the shaft contact surface and that of the connecting member contact surface as a result of abrasion of the shaft 15 and the connecting member 17. In the salt water test, after lapse of two weeks (2W), occurrence of rust in the surface of the shaft 15 was not observed.

In Comparative Example 1, a slight change arose in the appearance of the shaft contact surface and that of the connecting member contact surface as a result of abrasion of the shaft 15 and the connecting member 17. However, no decrease in the number of rotations of the shaft arose. Moreover, as a result of the shaft and the connecting member having been subjected to the drive test 20,000 times, no decrease in the number of rotations of the shaft arose. However, a further change in the appearance as a result of abrasion was observed. In the salt water test, after lapse of five hours, occurrence of small rust on the shaft 15 was observed.

In Comparative Example 2, the shaft 15 failed to rotate after having operated tens of times. In the salt water test, occurrence of rust in the surface of the shaft 15 was not observed after lapse of two weeks (2W).

<Results>

Examples 1 through 3 yielded superior results in the drive and salt water tests. Since DLC coating is slightly inferior in terms of an anti-impact characteristic, a DLC layer is considered to be subjected to exfoliation during the course of driving operation for reasons of impact. In consideration of this point, if the combination described in connection with Example 1 is employed, an anti-friction characteristic, a low friction characteristic, and a non-adhesion characteristic can be ensured even when the DLC layer on the surface of the shaft 15 is exfoliated, because the connecting member 17 has been subjected to pionite treatment.

As has been described, the invention can prevent occurrence of rust or adhesion of metal, thereby enabling stable rotation of a rotating shaft.

What is claimed is:

1. A corneal surgical apparatus for incising a cornea of a patient's eye in a layered form, comprising:
a rotating shaft; and
a bearing rotatably supporting the rotating shaft,
wherein at least one of a surface of the rotating shaft and an inner wall surface of the bearing has been subjected to a coating treatment or a surface modification treatment.

2. The apparatus of claim 1, wherein the coating treatment includes a DLC coating treatment.

3. The apparatus of claim 2, wherein the coating treatment applies a film having a thickness of 0.5 to 1.5 μm onto the at least one of the rotating shaft surface and the bearing inner wall surface.

4. The apparatus of claim 1, wherein the coating treatment applies a film having a friction coefficient equal to or less than 0.7 onto the at least one of the rotating shaft surface and the bearing inner wall surface.

5. The apparatus of claim 1, wherein the surface modification treatment includes a carbon solution diffusion processing.

6. The apparatus of claim 1, wherein at least one of the rotating shaft and the bearing is made of stainless steel.

7. A corneal surgical apparatus for incising a cornea of a patient's eye in a layered form, comprising:
a rotating shaft having a surface that has been subjected to a DLC coating treatment; and
a bearing rotatably supporting the rotating shaft, the bearing having an inner wall surface that has been subjected to a surface modification treatment.
